(19) United States
(12) Patent Application Publication
(10) Pub. No.: US 2022/0386896 A1
(43) Pub. Date: Dec. 8, 2022

(54) WALKING STEADINESS USER INTERFACES

(71) Applicant: Apple Inc., Cupertino, CA (US)

(72) Inventors: Nicholas D. FELTON, Sunnyvale, CA (US); Edith M. ARNOLD, Redwood City, CA (US); Robert CARLSEN, San Jose, CA (US); Dmitri CAVANDER, San Francisco, CA (US); Matthew W. CROWLEY, Sunnyvale, CA (US); Heather E. DANIEL, San Jose, CA (US); Eamon F. GILRAVI, San Francisco, CA (US); Ruchi N. GOSWAMI, Sunnyvale, CA (US); James D. KRETLOW, The Woodlands, TX (US); Catherine B. TING, San Francisco, CA (US); Adeeti V. ULLAL, Los Altos, CA (US); Mariah W. WHITMORE, San Diego, CA (US)

(21) Appl. No.: 17/752,802

(22) Filed: May 24, 2022

Related U.S. Application Data
(60) Provisional application No. 63/197,419, filed on Jun. 6, 2021.

(51) Int. Cl. A61B 5/11; A61B 5/00; G16H 20/30

(52) U.S. Cl. CPC A61B 5/112; A61B 5/743; A61B 5/7435; A61B 5/7275; G16H 20/30

(57) ABSTRACT

The present disclosure generally relates to user interfaces for managing walking steadiness data. In some embodiments, a walking steadiness state is determined based on detected movement. When a first set of conditions are met, a notification is displayed. When a second set of conditions is met, the notification is not displayed.

### CROSS-REFERENCE TO RELATED APPLICATIONS

This application claims priority to U.S. Provisional Patent Application No. 63/197,419, entitled “WALKING STEADINESS USER INTERFACES,” filed Jun. 6, 2021, which is hereby incorporated by reference in its entirety for all purposes.

### FIELD

The present disclosure relates generally to computer user interfaces, and more specifically to techniques for managing walking steadiness data.

### BACKGROUND

Computers can be used to provide a user with feedback about the health of the user. For example, a computer can monitor a user's heartrate and provide the user with feedback about the user's heartrate. For another example, a computer can be used to monitor a user's blood oxygen level and provide the user with feedback about the user's blood oxygen level.

### BRIEF SUMMARY

Some techniques for managing walking steadiness data using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for managing walking steadiness data. Such methods and interfaces optionally complement or replace other methods for managing walking steadiness data. Such methods and interfaces reduce the cognitive burden on a user and produce a more efficient human-machine interface. For battery-operated computing devices, such methods and interfaces conserve power and increase the time between battery charges.

In accordance with some embodiments, a method is described. The method is performed at a computer system that is in communication with one or more output generation components and one or more input devices. The method comprises: detecting, via the one or more input devices, movement data associated with movement of a user of the computer system; determining a walking steadiness state based on the detected movement data; and in response to determining the walking steadiness state: in accordance with a determination that a first set of conditions is met, outputting, via the one or more output generation components, a notification associated with the walking steadiness state; and in accordance with a determination that a second set of conditions, different from the first set of conditions, is met, forgoing outputting a notification associated with the walking steadiness state.

In accordance with some embodiments, a non-transitory computer-readable storage medium is described. The non-transitory computer-readable storage medium stores one or more programs configured to be executed by one or more processors of a computer system that is in communication with one or more output generation components and one or more input devices, the one or more programs including instructions for: detecting, via the one or more input devices, movement data associated with movement of a user of the computer system; determining a walking steadiness state based on the detected movement data; and in response to determining the walking steadiness state: in accordance with a determination that a first set of conditions is met, outputting, via the one or more output generation components, a notification associated with the walking steadiness state; and in accordance with a determination that a second set of conditions, different from the first set of conditions, is met, forgoing outputting a notification associated with the walking steadiness state.

In accordance with some embodiments, a transitory computer-readable storage medium is described. The transitory computer-readable storage medium stores one or more programs configured to be executed by one or more processors of a computer system that is in communication with one or more output generation components and one or more input devices, the one or more programs including instructions for: detecting, via the one or more input devices, movement data associated with movement of a user of the computer system; determining a walking steadiness state based on the detected movement data; and in response to determining the walking steadiness state: in accordance with a determination that a first set of conditions is met, outputting, via the one or more output generation components, a notification associated with the walking steadiness state; and in accordance with a determination that a second set of conditions, different from the first set of conditions, is met, forgoing outputting a notification associated with the walking steadiness state.

In accordance with some embodiments, a computer system is described. The computer system is configured to communicate with one or more output generation components and one or more input devices, and comprises: one or more processors; and memory storing one or more programs configured to be executed by the one or more processors, the one or more programs including instructions for: detecting, via the one or more input devices, movement data associated with movement of a user of the computer system; determining a walking steadiness state based on the detected movement data; and in response to determining the walking steadiness state: in accordance with a determination that a first set of conditions is met, outputting, via the one or more output generation components, a notification associated with the walking steadiness state; and in accordance with a determination that a second set of conditions, different from the first set of conditions, is met, forgoing outputting a notification associated with the walking steadiness state.

In accordance with some embodiments, a computer system is described. The computer system is configured to communicate with one or more output generation components and one or more input devices, and comprises: means for detecting, via the one or more input devices, movement data associated with movement of a user of the computer system; means for determining a walking steadiness state based on the detected movement data; and means for in response to determining the walking steadiness state: in accordance with a determination that a first set of conditions is met, outputting, via the one or more output generation components, a notification associated with the walking steadiness state; and in accordance with a determination that a second set of conditions, different from the first set of conditions, is met, forgoing outputting a notification associated with the walking steadiness state.

In accordance with some embodiments, a computer program product is described. The computer program product comprises one or more programs configured to be executed by one or more processors of a computer system that is in communication with one or more output generation components and one or more input devices, the one or more programs including instructions for: detecting, via the one or more input devices, movement data associated with movement of a user of the computer system; determining a walking steadiness state based on the detected movement data; and in response to determining the walking steadiness state: in accordance with a determination that a first set of conditions is met, outputting, via the one or more output generation components, a notification associated with the walking steadiness state; and in accordance with a determination that a second set of conditions, different from the first set of conditions, is met, forgoing outputting a notification associated with the walking steadiness state.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for managing walking steadiness data, thereby increasing the effectiveness, efficiency, and user satisfaction with such devices. Such methods and interfaces may complement or replace other methods for managing walking steadiness data.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3 is a block diagram of an exemplary multifunction device with a display and a touch-sensitive surface in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6S illustrate exemplary user interfaces for managing walking steadiness data, in accordance with some embodiments.

FIG. 7 depicts a flow diagram illustrating a method for managing walking steadiness data, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for managing walking steadiness data. Such techniques can reduce the cognitive burden on a user who accesses walking steadiness data, thereby enhancing productivity. Further, such techniques can reduce processor and battery power otherwise wasted on redundant user inputs.

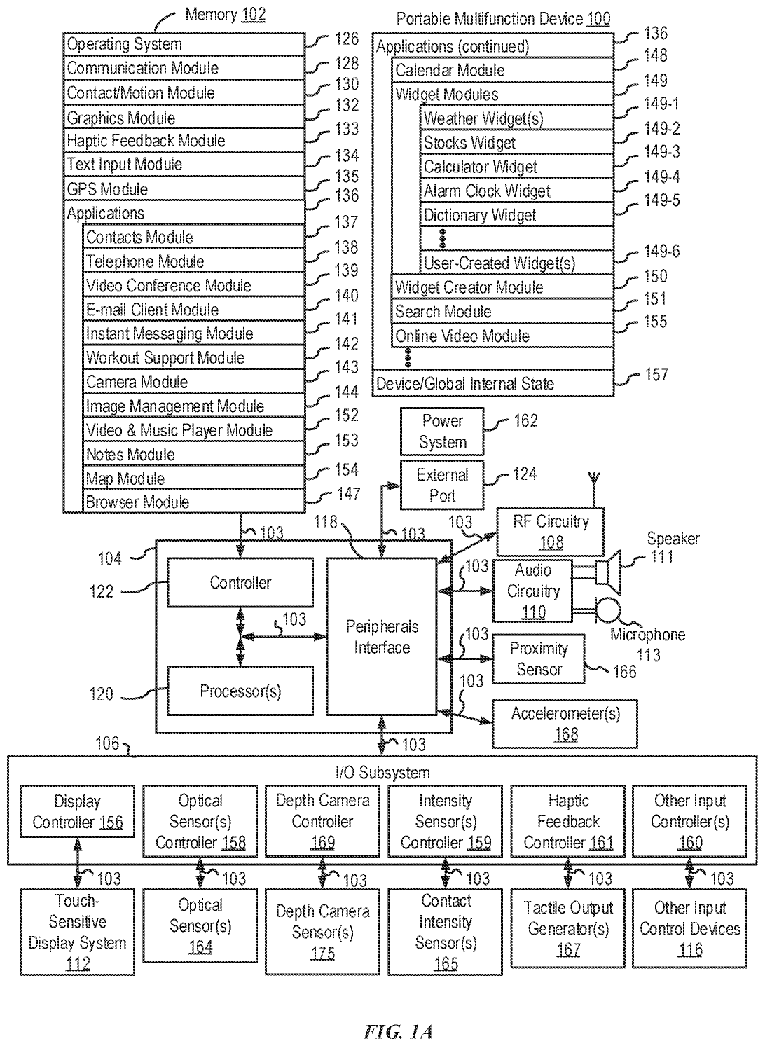

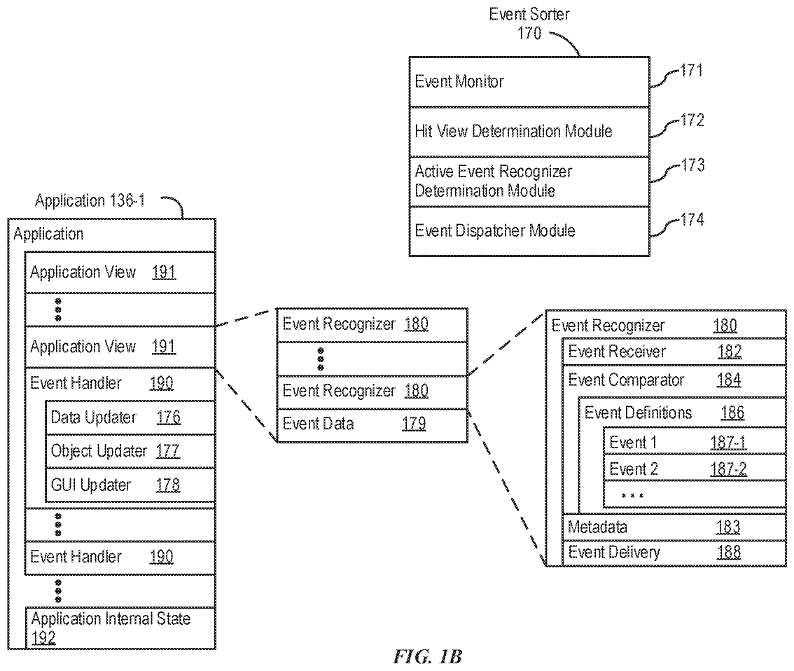

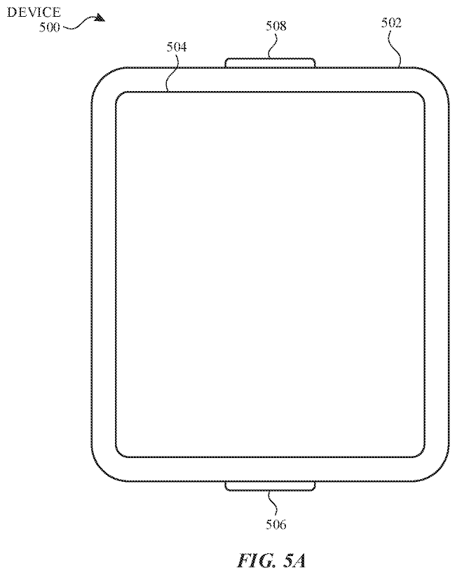

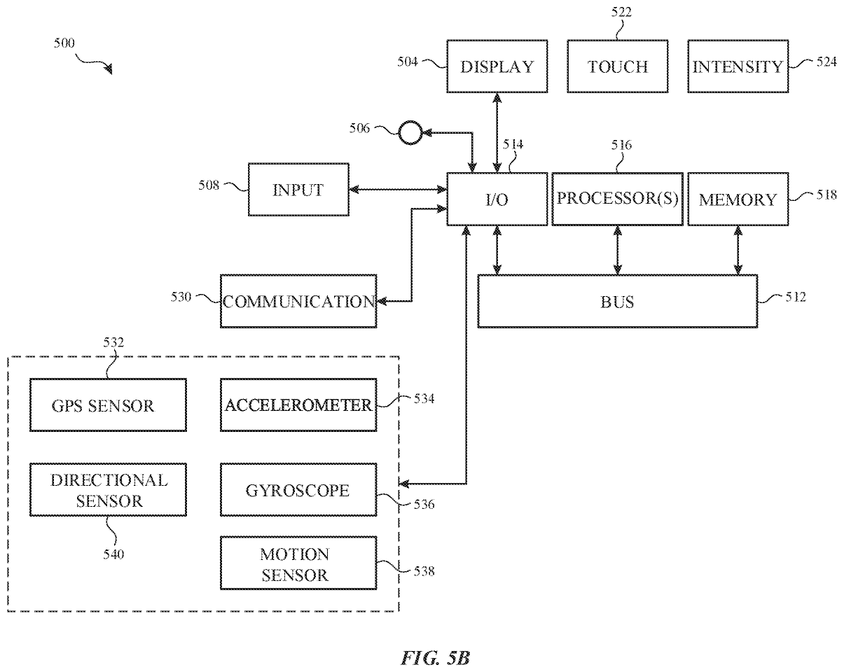

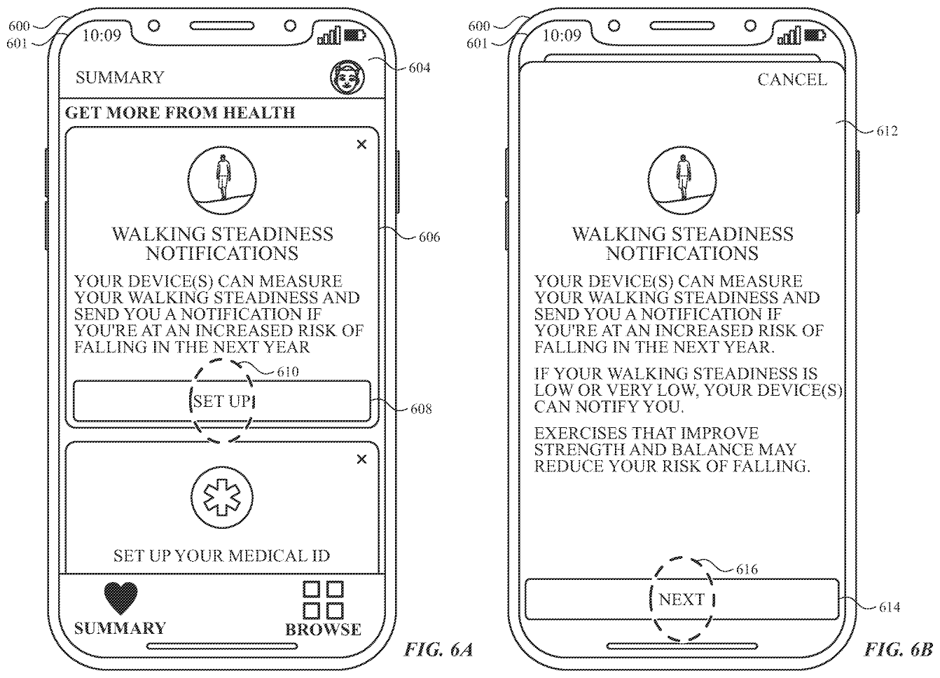

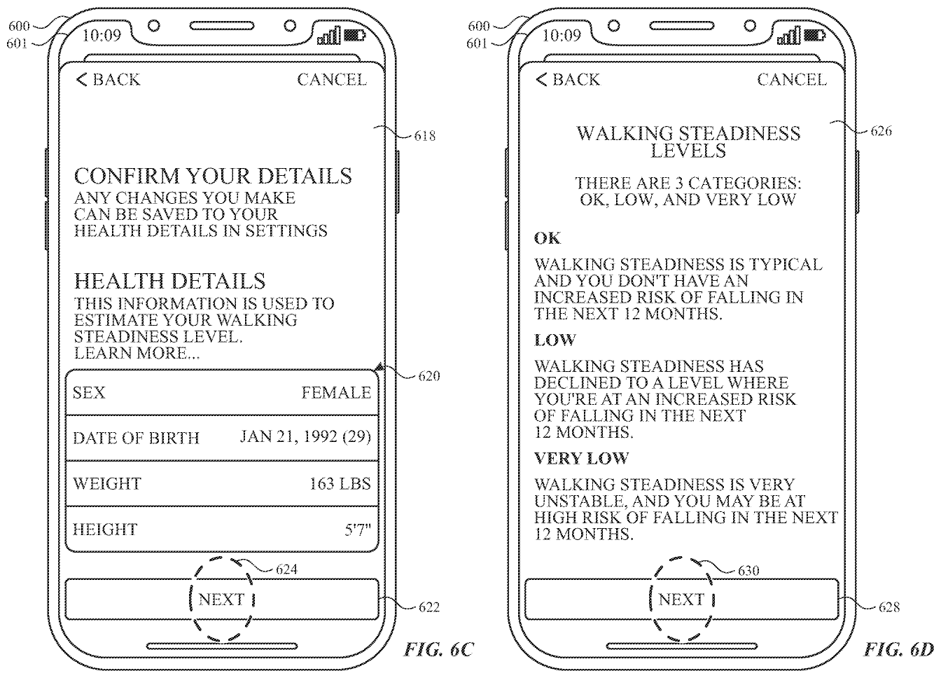

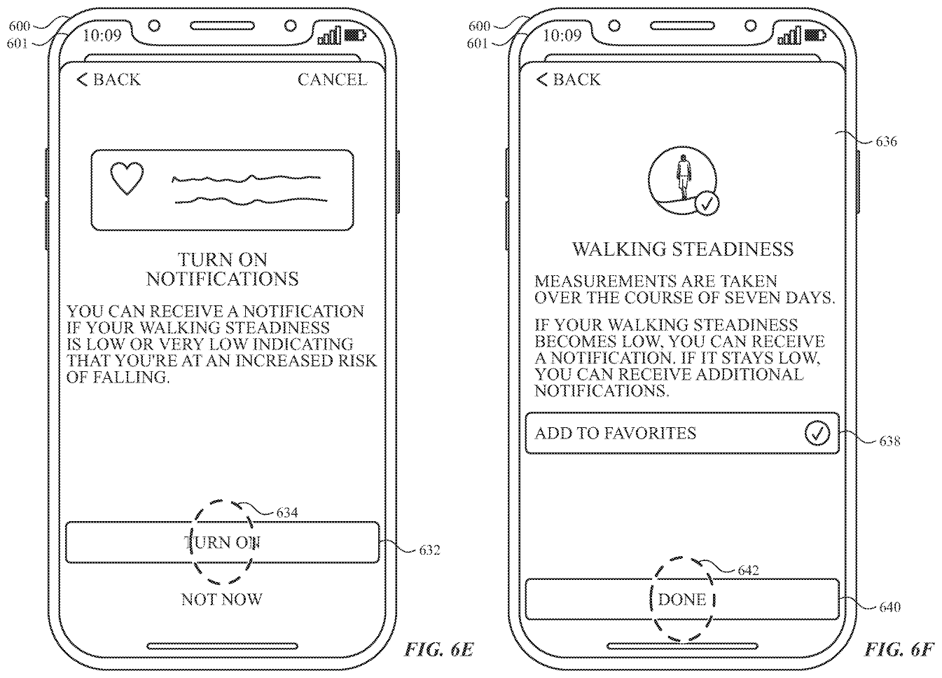

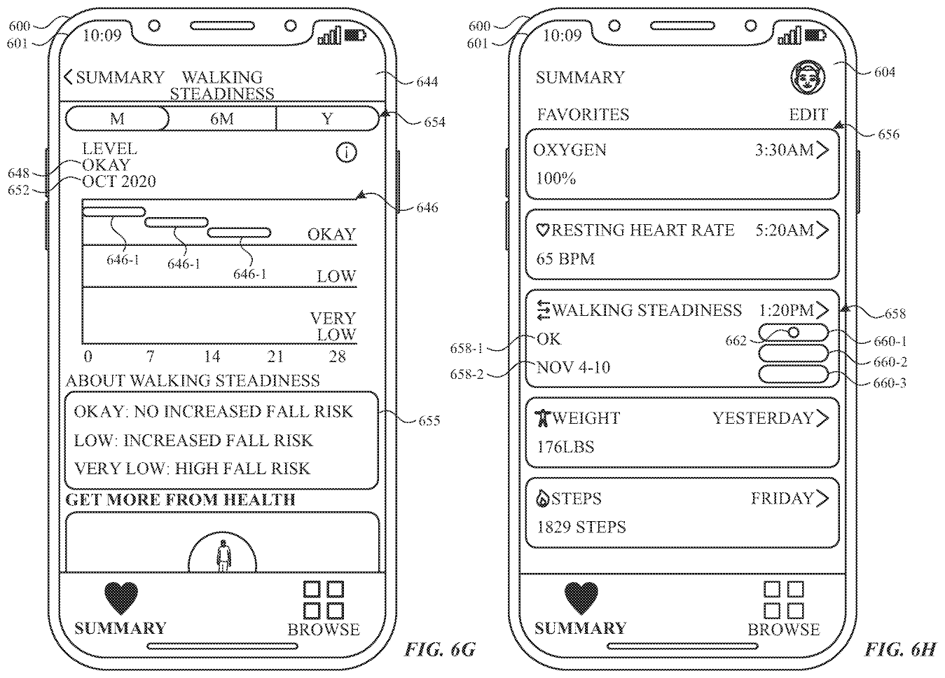

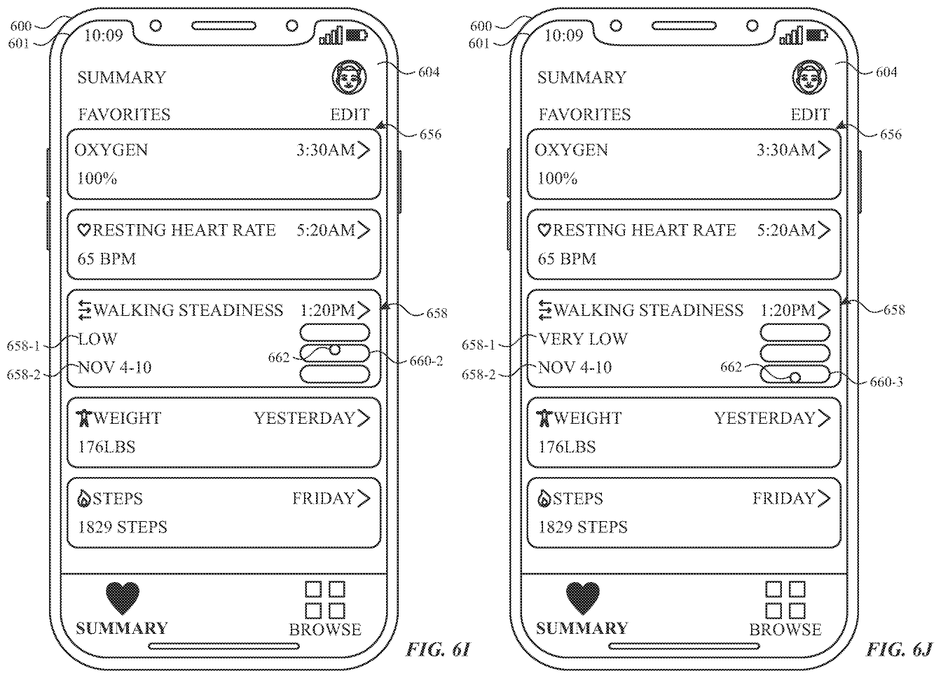

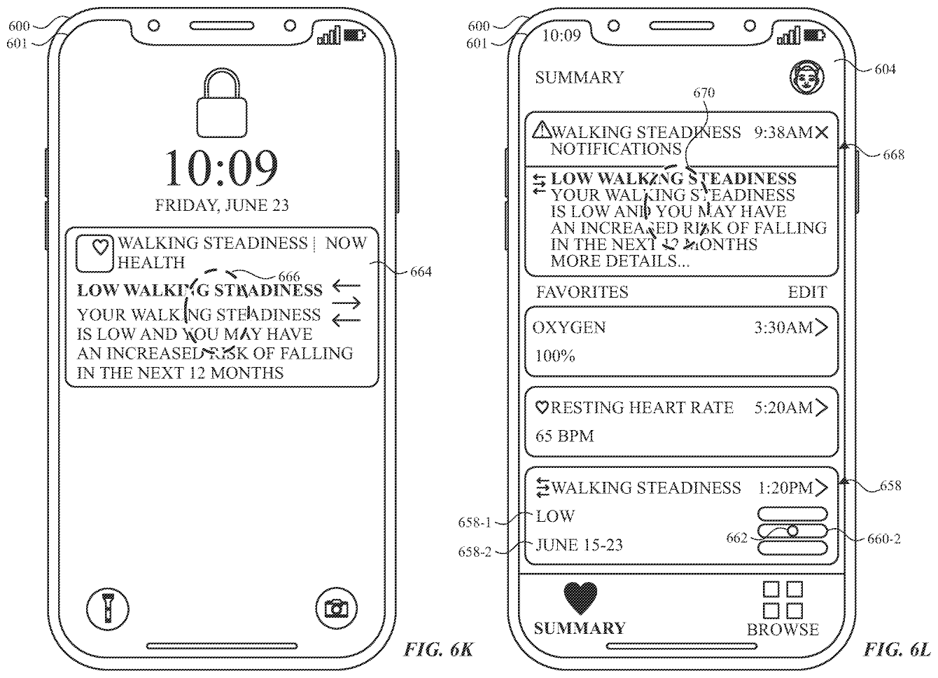

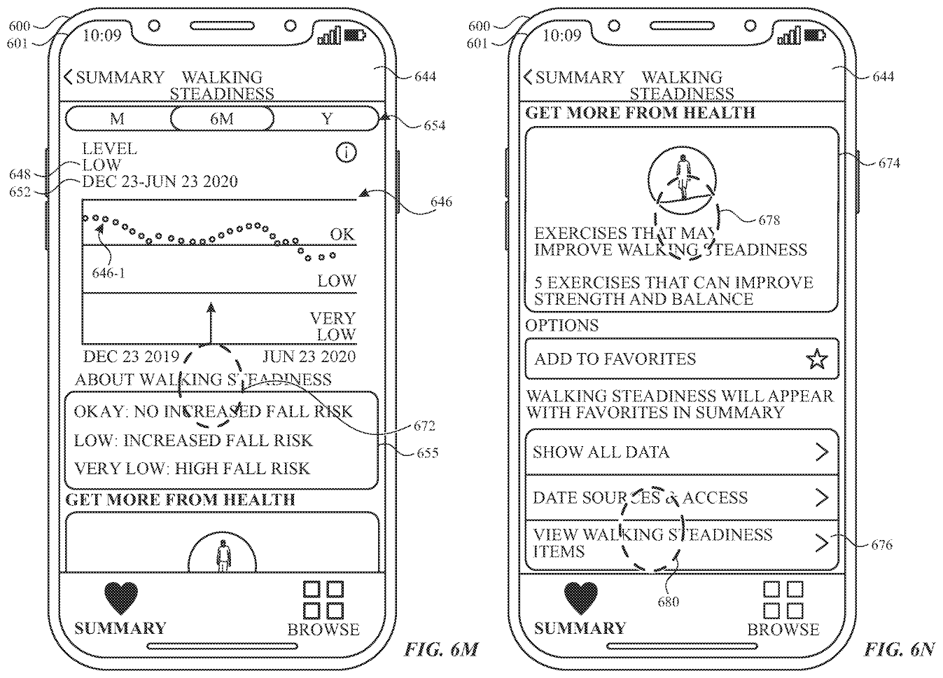

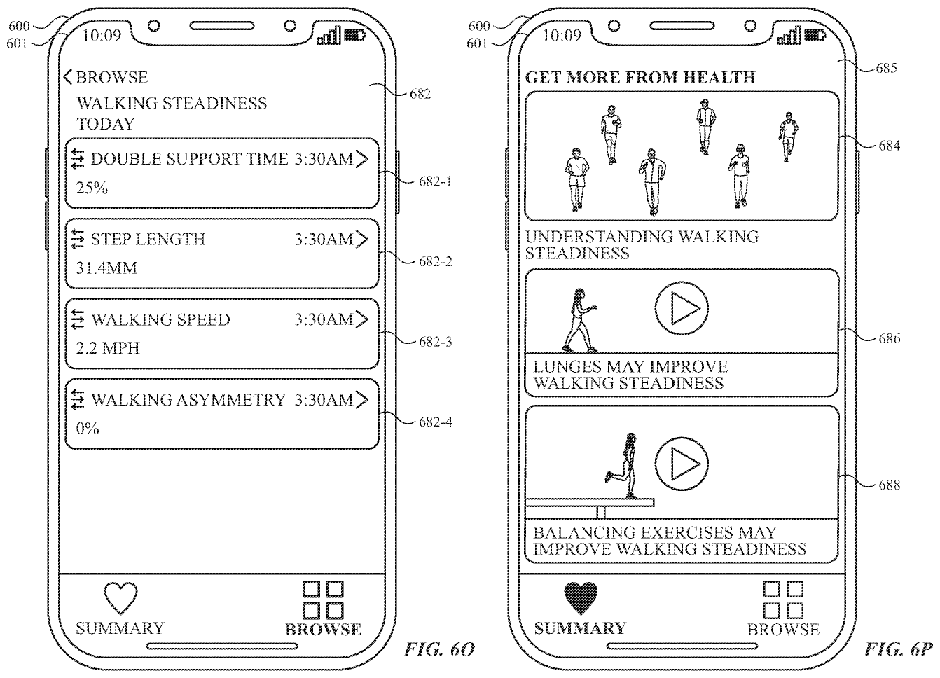

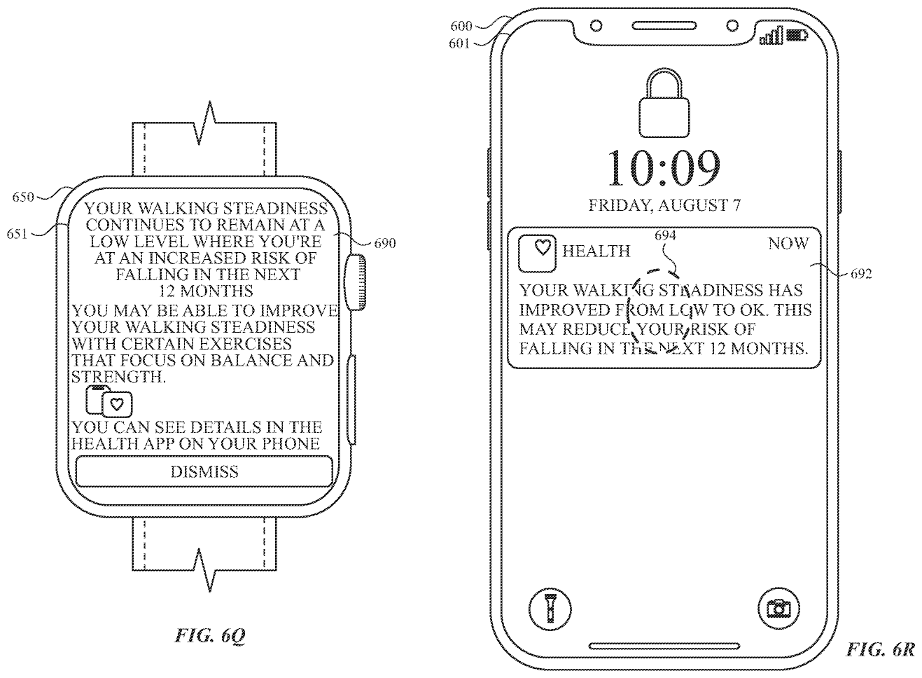

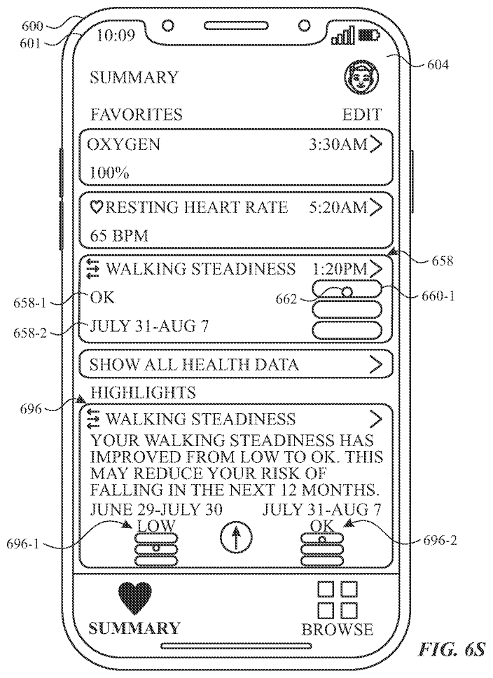

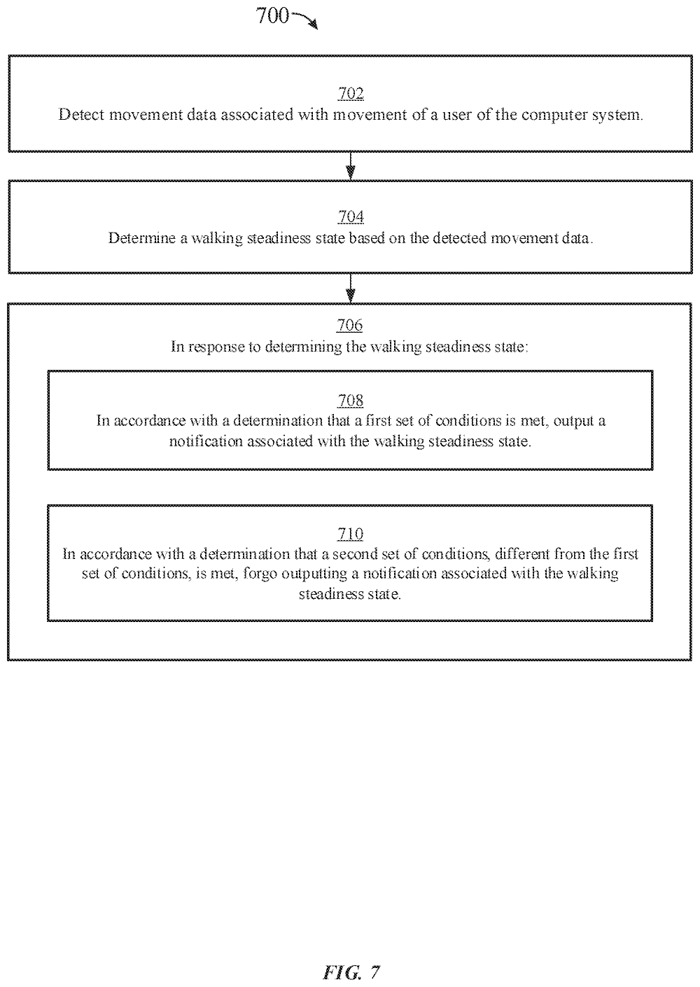

Below, FIGS. 1A-1B, 2, 3, 4A-4B, and 5A-5B provide a description of exemplary devices for performing the techniques for managing walking steadiness data. FIGS. 6A-6S illustrate exemplary user interfaces for managing walking steadiness data. FIG. 7 is a flow diagram illustrating methods of managing walking steadiness data in accordance with some embodiments. The user interfaces in FIGS. 6A-6S are used to illustrate the processes described below, including the processes in FIG. 7.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, Calif. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

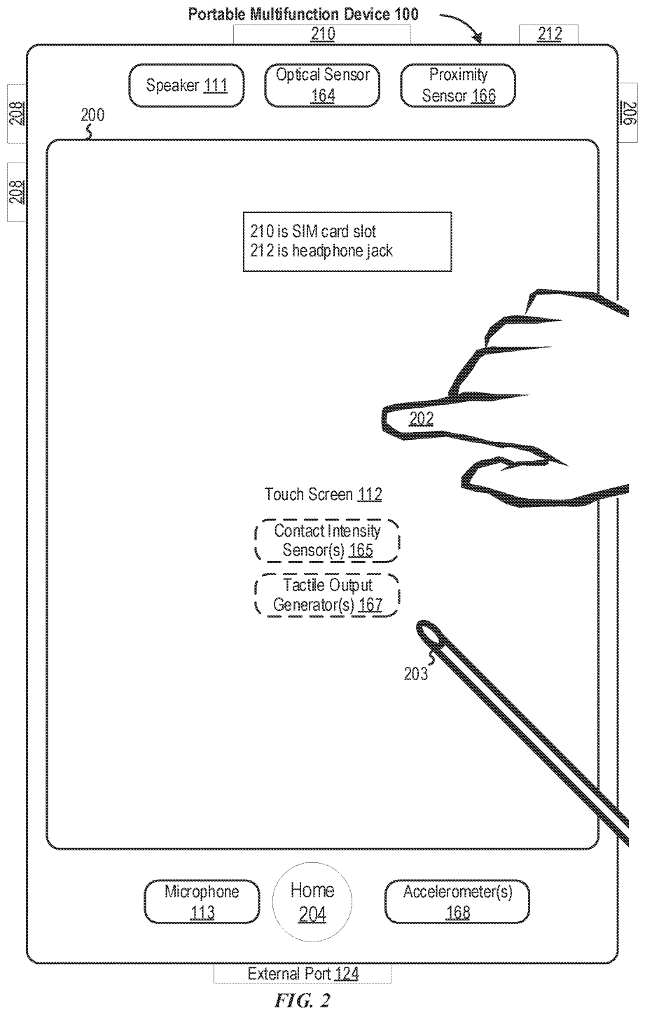

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, Calif.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

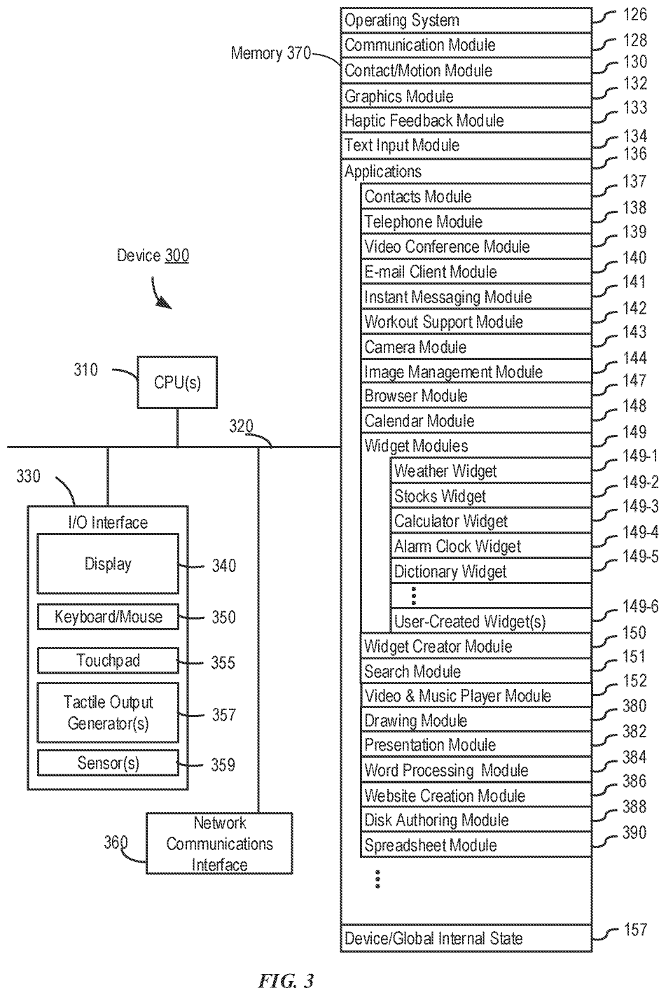

In some embodiments, the software components stored in memory 102 include operating system 126, communication module (or set of instructions) 128, contact/motion module (or set of instructions) 130, graphics module (or set of instructions) 132, text input module (or set of instructions) 134, Global Positioning System (GPS) module (or set of instructions) 135, and applications (or sets of instructions) 136. Furthermore, in some embodiments, memory 102 (FIG. 1A) or 370 (FIG. 3) stores device/global internal state 157, as shown in FIGS. 1A and 3. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, iOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts 137, e-mail 140, IM 141, browser 147, and any other application that needs text input).

GPS module 135 determines the location of the device and provides this information for use in various applications (e.g., to telephone 138 for use in location-based dialing; to camera 143 as picture/video metadata; and to applications that provide location-based services such as weather widgets, local yellow page widgets, and map/navigation widgets).

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone 138, video conference module 139, e-mail 140, or IM 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo! Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3 is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3 is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Attention is now directed towards embodiments of user interfaces that are, optionally, implemented on, for example, portable multifunction device 100.

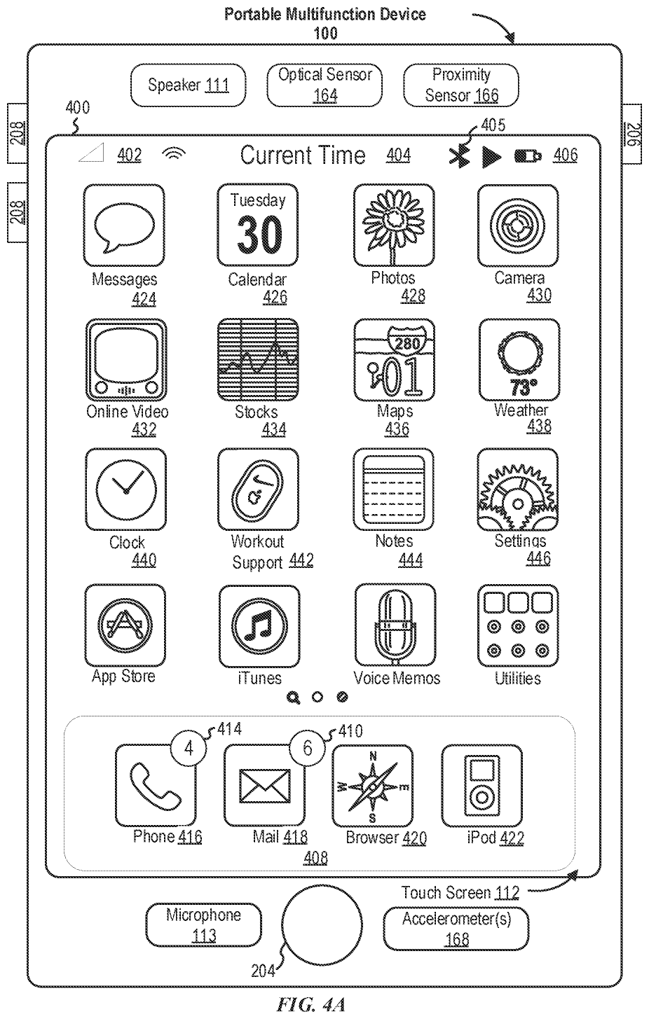

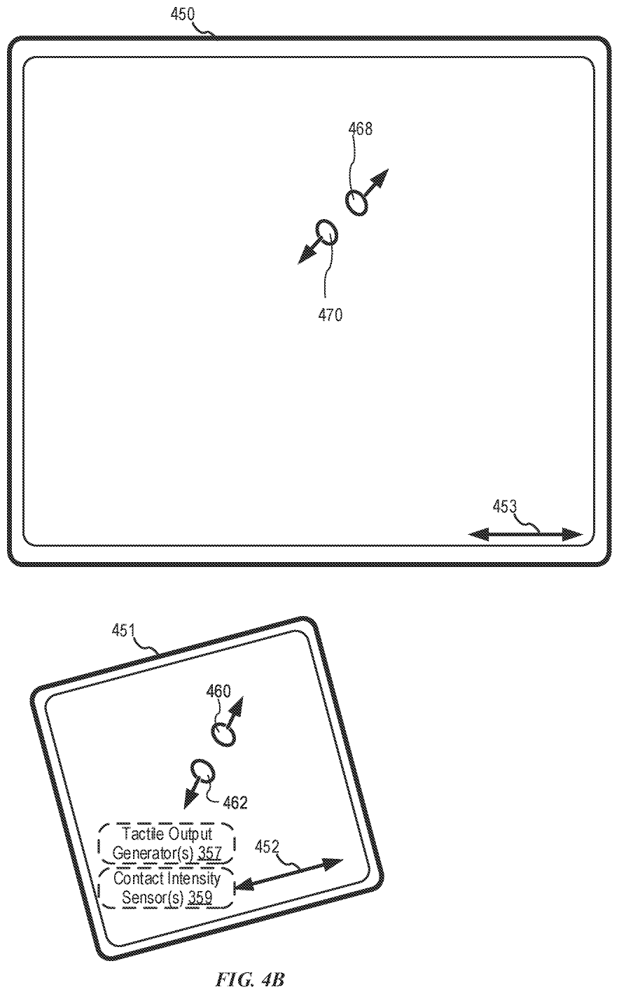

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3) with a touch-sensitive surface 451 (e.g., a tablet or touchpad 355, FIG. 3) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3. Device 500 has bus 512 that operatively couples I/O section 514 with one or more computer processors 516 and memory 518. I/O section 514 can be connected to display 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including process 700 (FIG. 7). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3 or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

Attention is now directed towards embodiments of user interfaces (“UI”) and associated processes that are implemented on an electronic device, such as portable multifunction device 100, device 300, or device 500.

FIGS. 6A-6S illustrate exemplary user interfaces for managing walking steadiness data, in accordance with some embodiments. The user interfaces in these figures are used to illustrate the processes described below, including the processes in FIG. 7.

FIG. 6A illustrates device 600, which includes one or more elements of devices 100, 300, or 500. For example, device 600 includes display 601 (e.g., a touch-sensitive display), which is displaying health interface 604, an interface for a health application operating at device 600. Health interface 604 includes walking steadiness platter 606, which provides information about walking steadiness notifications that can be displayed at, for example, device 600. Device 600 detects input 610 (e.g., a tap input detected via display 601) selecting setup option 608 and, in response, initiates a process for enabling walking steadiness notifications.

In FIG. 6B, device 600 displays information 612 notifying a user of device 600 about walking steadiness notifications that can be displayed to warn a user if they have an increased risk of falling due to detected low or very low levels of walking steadiness. Device 600 detects input 616 (e.g. a tap input) selecting next option 614 to continue with the process for enabling walking steadiness notifications (e.g., continue to health details interface 618 at FIG. 6C).

In FIG. 6C, device 600 displays health details interface 618, which prompts the user to confirm biometric data 620 used to determine a walking steadiness of the user. In some embodiments, the biometric data includes the user's sex, age, weight, height, and/or other biometric data for the user. Health details interface 618 optionally enables the user to enter (or update) the same biometric data for the user (e.g., by taping on “weight” and entering the user's current weight). Device 600 detects input 624 (e.g., a tap input) on next affordance 622 and, in response, displays interface 626, as shown in FIG. 6D.

Interface 626 of FIG. 6D presents information for three levels of walking steadiness: OK, low, and very low. A walking steadiness level of “OK” is used to indicate that the user has a typical and/or normal walking steadiness and, therefore, is not at an increased risk of falling. A walking steadiness level of “low” is used to indicate that the user's walking steadiness is below the normal walking steadiness (e.g., has declined) and, therefore, the user is at an increased risk of falling. A walking steadiness level of “very low” is used to indicate that the user's walking steadiness is very unstable and, therefore, the user is at a high risk of falling. In some embodiments, the risk of falling is associated with (e.g., calculated for) a period of time in the future such as, for example, the next month, two months, six months, or year.

In FIG. 6D, device 600 detects input 630 (e.g., a tap input) on next affordance 628 and, in response, displays the user interface depicted in FIG. 6E for enabling walking steadiness notifications. Device 600 detects input 634 on enable option 632 and, in response, enables walking steadiness notifications. Alternatively, the “not now” option can be selected to forgo enabling walking steadiness notifications. In some embodiments, device 600 displays (e.g., in the health application or a settings application of device 600) a toggle for enabling or disabling walking steadiness notifications.

In FIG. 6F, device 600 displays interface 636 confirming that walking steadiness notifications are enabled, and providing option 638 to add walking steadiness information to a favorites section of the health application (e.g., via a tap input on option 638). Device 600 detects input 642 (e.g., a tap input) on done affordance 640 and, in response, displays walking steadiness interface 644, as shown in FIG. 6G.

Walking steadiness interface 644 of FIG. 6G is an interface for managing various aspects of walking steadiness data. Walking steadiness interface 644 includes chart 646 including walking steadiness data 646-1 and level indicator 648, which indicates that the user's current walking steadiness is at the “OK” level for date range 652, which is October of 2020. In some embodiments, one or more instances of walking steadiness data 646-1 can be selected to display an indication of the walking steadiness level for a date corresponding to the selected instance(s) of the walking steadiness data. In the embodiment depicted in FIG. 6G, the data in chart 646 represents walking steadiness data that has been collected (e.g., measured) before walking steadiness notifications were enabled for device 600 (e.g., before device 600 detects input 610 at FIG. 6A). In some embodiments, the walking steadiness data is used to establish a walking steadiness baseline for the user in order to determine whether the user's current walking steadiness has deviated from the baseline, and whether the deviation is such that the user's current walking steadiness is low or very low. In FIG. 6G, chart 646 depicts walking steadiness data 646-1 collected over a one-month period, and displayed based on a week-by-week basis. The vertical location of each bar of chart 646 indicates the walking steadiness level for each week and where within the walking steadiness level (e.g., top of “OK” vs. bottom of “OK”) the walking steadiness resides. The horizontal location of each bar of chart 646 indicates the period for which the bar is presenting information (e.g., which week of the month). However, chart 646 can be updated to show the walking steadiness data collected for different periods of time by selecting the six-month or one-year options from date range options 654.

Walking steadiness interface 644 includes risk information 655, which indicates a fall risk associated with each of the respective walking steadiness levels. For example, the OK walking steadiness level corresponds to no increased risk of falling, the low walking steadiness level corresponds to an increased risk of falling (e.g., within the next 12 months), and the very low walking steadiness level corresponds to a high risk of falling (e.g., within the next 12 months).

FIGS. 6H-6J depict user interfaces of example walking steadiness status indicators for different walking steadiness levels. In FIGS. 6H-6J, device 600 displays health interface 604, which includes favorites section 656. Included in favorites section 656 is walking steadiness status indicator 658, which includes level indicator 658-1 and date range 658-2. Walking steadiness status indicator 658 in FIG. 6H shows that the walking steadiness is OK from November 4^{th} to November 10^{th} (e.g., the most recent period). Walking steadiness status indicator 658 in FIG. 6I shows that the walking steadiness is low from November 4^{th} to November 10^{th}. Walking steadiness status indicator 658 in FIG. 6J shows that the walking steadiness is very low from November 4^{th} to November 10^{th}.

Walking steadiness status indicator 658 also includes a graphical depiction of the current walking steadiness level, which is represented by dot 662 positioned within a bar 600 that corresponds to one of the walking steadiness levels. Bar 660-1 corresponds to the OK walking steadiness level, bar 660-2 corresponds to the low walking steadiness level, and bar 660-3 corresponds to the very low walking steadiness level. In some embodiments, dot 662 is displayed at a location (e.g., a vertically or horizontally offset position) within a respective bar to indicate a more specific feedback about walking steadiness within the corresponding walking steadiness level. When the walking steadiness is within an upper range of a respective walking steadiness level, dot 662 is positioned at an upper edge of the corresponding bar, when the walking steadiness is within a middle range of the respective walking steadiness level, dot 662 is positioned in the middle of the bar, and when the walking steadiness is within a lower range of the respective walking steadiness level, dot 662 is positioned at a lower edge of the bar. For example, in FIG. 6H, dot 662 is located in the middle of bar 660-1, indicating that the current walking steadiness is in the middle range of the OK walking steadiness level. In FIG. 6I, dot 662 is located at the upper edge of bar 660-2, indicating that the current walking steadiness is in the upper range of the low walking steadiness level. In FIG. 6J, dot 662 is located at the lower edge of bar 660-3, indicating that the current walking steadiness is in the lower range of the very low walking steadiness level.

In some embodiments, device 600 displays notifications based on a determined walking steadiness level, maintaining a particular walking steadiness level, and/or changes in a walking steadiness level. For example, the notifications can be displayed to inform a user that their walking steadiness has decreased and/or is at a level where the user is at an increased risk of falling. In some embodiments, device 600 displays notifications to inform the user that their walking steadiness level remains low or very low. In some embodiments, device 600 displays notifications to inform the user that their walking steadiness has increased. Examples of such notifications are discussed in greater detail below with reference to FIGS. 6K-6S. In some embodiments, device 600 displays notifications to inform the user that they have not completed a recommended exercise.

In FIG. 6K, device 600 displays notification 664 informing the user that their walking steadiness level is low (e.g., based on the user's walking steadiness level having been maintained at the low walking steadiness level for a threshold duration of time, such as 3 days, 5 days, or 7 days). In this embodiment, the user's walking steadiness has decreased from the OK level to the low level and, in response, device 600 notifies the user that they are now at an increased risk of falling. In some embodiments, device 600 displays a similar notification to inform the user that their walking steadiness is very low, for example, if the user's walking steadiness decreases from the low level to the very low level (e.g., and is maintained at the very low walking steadiness level for a threshold duration of time), or from the OK level to the very low level (e.g., and is maintained at the very low walking steadiness level for a threshold duration of time).

Device 600 detects input 666 (e.g., a tap input) on notification 664 and, in response, displays health interface 604, as shown in FIG. 6L. Health interface 604 is updated (e.g., in response to determining that the user's walking steadiness is at the low level) to include notification 668, which is similar to notification 664. Health interface 604 includes walking steadiness status indicator 658, which is updated to show that the user's walking steadiness is at the low level from June 15-23. Accordingly, dot 662 is displayed positioned in bar 660-2. In some embodiments, notification 668 and/or walking steadiness status indicator 658 can be selected (e.g., via a tap input) to display walking steadiness interface 644. For example, in FIG. 6L, device 600 detects input 670 (e.g., a tap input) on notification 668 and, in response, displays walking steadiness interface 644, as shown in FIG. 6M.

In FIG. 6M, chart 646 is updated (e.g., based on a tap input on “6M” in 654) to show walking steadiness data 646-1 recorded during the prior six months, from Dec. 23, 2019 to Jun. 23, 2020. The data in chart 646 indicates that the user's walking steadiness decreased from the OK level to the low level around June 15^{th}, and has remained in the low level since. Device 600 detects input 672 (e.g., a swipe up input), which is a scroll gesture for scrolling walking steadiness interface 644. In response to detecting input 672, device 600 scrolls walking steadiness interface 644, as shown in FIG. 6N.

In FIG. 6N, device 600 displays walking steadiness interface 644 having walking improvement platter 674 and walking steadiness data options, including walking steadiness items 676. As shown in FIG. 6N, device 600 detects input 678 (e.g., a tap input) on walking improvement platter 674 and/or input 680 (e.g., a tap input) on walking steadiness items 676. In response to input 680, device 600 displays walking metrics interface 682 (as shown in FIG. 6O), which depicts various data metrics that are optionally used by device 600 to determine the user's walking steadiness. For example, double support time 682-1 provides an indication of the amount of time that both feet are in contact with the ground simultaneously, step length 682-2 indicates the length of the user's step, walking speed 682-3 indicates the average speed at which the user walks, and walking asymmetry 682-4 indicates a percentage of time that the user's steps with one foot are faster or slower than with the other foot.

In FIG. 6N, in response to input 678 on walking improvement platter 674, device 600 displays walking steadiness improvement interface 685, as shown in FIG. 6P. Walking steadiness improvement interface 685 provides content and information (e.g., videos, PDFs, health articles, and/or links to applications) that can be used to improve walking steadiness. For example, item 684 is selectable (e.g., via a tap input) to display additional information such as articles, PDFs, and/or videos related to walking steadiness and/or techniques for improving walking steadiness. Videos 686 and 688 are exercise videos that can be selected (e.g., via a tap input) to view exercises for improving walking steadiness. For example, video 686 can be selected (e.g., via a tap input) to display a video that teaches the user to perform lunges in order to improve walking steadiness. Video 688 can be selected (e.g., via a tap input) to display a video that teaches the user to perform balancing exercises that improve walking steadiness. In some embodiments, videos 686 and/or 688 can be launched in the health application or in a separate application such as, for example, an application associated with an exercise/fitness service.

In some embodiments, device 600 updates and/or modifies the content provided in walking steadiness improvement interface 685 based on various factors such as, for example, the user's current walking steadiness (e.g., most recent walking steadiness level), changes and/or patterns in the user's walking steadiness, improvements in walking steadiness, degradation of the user's walking steadiness, completion of various exercises, and/or failure to complete various exercises. In some embodiments, device 600 can remind the user to complete exercises. In some embodiments, device 600 incorporates various walking steadiness improvement exercises into different workout programs or routines such as, for example, a strength training workout.

In some embodiments, walking steadiness notifications can be displayed at other devices or computer systems (e.g., of the user of device 600). For example, in FIG. 6Q, device 650 (e.g., a smartwatch of the user of device 600) displays, via display screen 651 (e.g., a touch-sensitive display), notification 690 informing the user that their walking steadiness remains at the low (or very low) level. In some embodiments, devices (e.g., device 600 and/or device 651) display notifications if the user's walking steadiness remains at a low or very low level for an extended period of time such as, for example, two weeks, three weeks, three months, four months, etc. In some embodiments, device 650 can be used to view content provided in health interface 604 and/or walking steadiness interface 644.

In FIG. 6R, device 600 displays notification 692 informing the user that their walking steadiness has improved from the low level to the OK level. In some embodiments, device 600 displays notifications when the user's walking steadiness improves from very low to low, or from very low to OK. Device 600 detects input 694 (e.g., a tap input) on notification 692 and, in response, displays heath interface 604, as shown in FIG. 6S.

In FIG. 6S, device 600 has updated health interface 604 to indicate that the user's walking steadiness has improved to the OK level from July 31^{st} to August 7^{th}. For example, walking steadiness status indicator 658 is updated to show dot 662 at the bottom edge of bar 660-1. In addition, health interface 604 includes walking steadiness improvement information 696 showing prior walking steadiness level 696-1 having the low level from June 29^{th} to July 30^{th}, and current walking steadiness level 696-2 having the OK level from July 31^{st} to August 7^{th}. In some embodiments, walking steadiness status indicator 658 and/or walking steadiness improvement information 696 can be selected (e.g., via a tap input) to display walking steadiness interface 644, which in this embodiment, would show chart 646 with data 646-1 depicting an increase in the user's walking steadiness level from the low level to the OK level.

FIG. 7 is a flow diagram illustrating a method for managing walking steadiness data using a computer system in accordance with some embodiments. Method 700 is performed at a computer system (e.g., 100, 300, 500, 600, and/or 650) (e.g., a smartphone, a tablet, a wearable device (e.g., a smartwatch), a desktop or laptop computer) that is configured to communicate with one or more output generation components (e.g., 601, 651) (e.g., a display generation component, a touch-sensitive display system, a speaker, a bone conduction audio output device, a tactile output generator, a projector, and/or a holographic display) and one or more input devices (e.g., 601, 651) (e.g., a touch-sensitive surface, a keyboard, mouse, trackpad, one or more optical sensors for detecting gestures, one or more capacitive sensors for detecting hover inputs, accelerometer, gyroscope, inertial measurement components, and/or motion sensor). In some embodiments, the one or more input devices includes an accelerometer, gyroscope, inertial measurement components, and/or motion sensor of an external electronic device with which the computer system is in communication (e.g., a smart watch worn by the user collects movement data associated with movement of the user and the computer system receives the movement data). Some operations in method 700 are, optionally, combined, the orders of some operations are, optionally, changed, and some operations are, optionally, omitted.

As described below, method 700 provides an intuitive way for managing walking steadiness data. The method reduces the cognitive burden on a user for managing walking steadiness data, thereby creating a more efficient human-machine interface. For battery-operated computing devices, enabling a user to manage walking steadiness data faster and more efficiently conserves power and increases the time between battery charges.

In method 700, the computer system (e.g., 600 and/or 650) detects (702), via the one or more input devices (e.g., 601 and/or 651), movement data associated with movement (e.g., walking movement) of a user of the computer system (e.g., data generated from an input device (e.g., an accelerometer/gyroscope/inertial measurement component/motion sensor, such as of the computer system) that is used to record movement of the computer system, an external device, and/or a user as they move (e.g., walk)).

The computer system (e.g., 600 and/or 650) determines (704) a walking steadiness state (e.g., a likelihood (or range of likelihood) that the user is to experience (e.g., over a duration of time, such as the next 15 days, 30 days, 45 days, 3 months, 6 months, 1 year) a fall condition (e.g., falling down) due, at least in part, to the user's movement (e.g., walking)) based on (e.g., based at least on) the detected movement data.

At 706 of method 700, in response to determining the walking steadiness state, the computer system (e.g., 600 and/or 650) performs the following items. In accordance with a determination that a first set of conditions is met (e.g., including a condition that is met when the walking steadiness state is a first state, a condition that is met when the walking steadiness state is determined or maintained for a first duration, and/or a condition that is met when the walking steadiness state is different from a baseline walking steadiness state), the computer system outputs (708), via the one or more output generation components (e.g., 601 and/or 651), a notification (e.g., 664, 668, 690, 692, and/or 696) (e.g., displaying an alert, providing a tactile output, providing an audio output, and/or displaying a graphical user interface object, text, and/or a banner) associated with the walking steadiness state. In accordance with a determination that a second set of conditions, different from the first set of conditions, is met (e.g., including a condition that is met when the first set of conditions is not met, a condition that is met when the walking steadiness state is a second state different from the first state, a condition that is met when the walking steadiness state is determined or maintained for a second duration different from the first duration, and/or a condition that is met when the walking steadiness state is the same as a baseline walking steadiness state), the computer system forgoes (710) outputting a notification associated with the walking steadiness state. Outputting a notification associated with the walking steadiness state provides the user with feedback about the state of the computer system and the data that it has collected about the walking steadiness of the user, thereby providing improved visual feedback.

In some embodiments, the first set of conditions includes a state condition that is met when the walking steadiness state is a first state of walking steadiness (e.g., low walking steadiness state and/or very low walking steadiness state, the user is not steady when walking) that corresponds to a first range of walking steadiness scores (e.g., calculated using the movement data and/or biometric data (e.g., height, weight, gender, and/or age) of the user of the computer system) and the second set of conditions (optionally) includes a state condition that is met when the walking steadiness state is a second state of walking steadiness (e.g., high walking steadiness state, the user is steady when walking) that corresponds to a second range of walking steadiness scores that is different from the first range of walking steadiness scores. Outputting a notification associated with the walking steadiness state when the walking steadiness state is the first state provides the user with feedback about the state of the computer system and the data that it has collected about the walking steadiness of the user, thereby providing improved visual feedback.

In some embodiments, the first set of conditions includes a duration condition that is met when the walking steadiness state is maintained in a first state of walking steadiness (e.g., low walking steadiness state and/or very low walking steadiness state, the user is not steady when walking) (e.g., stays in the first state without changing states, stays in the first state without changing to the second state) for a first duration. Outputting a notification associated with the walking steadiness state when the walking steadiness state has been maintained for a first duration of time provides the user with feedback about the state of the computer system and the data that it has collected about the walking steadiness of the user, thereby providing improved visual feedback. In some embodiments, the computer system monitors the walking patterns of the user and determines a walking steadiness score, which is used to determine the walking steadiness state of the user. In some embodiments, the walking steadiness score and, therefore, the walking steadiness state of the user changes over time as the computer system detects movements (e.g., walking) of the user and characteristics of the movement. In some embodiments, once the walking steadiness state has remained in the first state for the first duration of time, the duration condition for the first set of conditions is met.

In some embodiments, the second set of conditions includes a duration condition that is met when the walking steadiness state has been maintained in a second state of walking steadiness (e.g., high walking steadiness state, the user is steady when walking) (e.g., stays in the second state without changing states, stays in the second state without changing to the first state) for a second duration (e.g., a duration of time that is less than the first duration). Forgoing outputting a notification associated with the walking steadiness state when the walking steadiness state has been maintained for a second duration of time provides the user with feedback about the state of the computer system and the data that it has collected about the walking steadiness of the user, thereby providing improved visual feedback. In some embodiments, once the walking steadiness state has remained in the second state for the second duration of time, the duration condition for the second set of conditions is met.

In some embodiments, subsequent to outputting, via the one or more output generation components (e.g., 601 and/or 651), the notification (e.g., displaying an alert, providing a tactile output, providing an audio output, and/or displaying a graphical user interface object, text, and/or a banner) associated with the walking steadiness state and in accordance with a determination that the walking steadiness state has remained in a first state of walking steadiness (e.g., low walking steadiness state and/or very low walking steadiness state, the user is not steady when walking) for a third duration (e.g., a duration different from the first duration and/or duration that is longer than the first duration) (and, optionally, in response to determining the walking steadiness state), the computer system (e.g., 600 and/or 650) provides (e.g., outputs) (e.g., displaying, via display generation component, an alert, providing a tactile output, providing an audio output, and/or displaying a graphical user interface object, text, and/or a banner) a second notification (e.g., 690) (e.g., a reminder that the walking steadiness state is the first state) that indicates that the walking steadiness state is the first state. Outputting a second notification that indicates that the walking steadiness state is the first state provides the user with feedback about the state of the computer system and the data that it has collected about the walking steadiness of the user, thereby providing improved visual feedback. In some embodiments, in response to determining the walking steadiness state and in accordance with a determination that a third set of conditions is met (e.g., including a condition that is met when the walking steadiness state is the first state, a condition that is met when the walking steadiness state is determined or maintained for a third duration (longer then the first duration)), the computer system outputs, via the one or more output generation components, a second notification (e.g., displaying an alert, providing a tactile output, providing an audio output, and/or displaying a graphical user interface object, text, and/or a banner) associated with the walking steadiness state (e.g., a reminder that the walking steadiness state is the second state).

In some embodiments, the first set of conditions includes a baseline condition that is met when the walking steadiness state is different from a baseline walking steadiness state, wherein the baseline walking steadiness state is a walking steadiness state of the user determined over a duration (e.g., over a duration prior to the computer system being enabled to provide notifications of walking steadiness state). Outputting a notification when the baseline condition is met provides the user with feedback about the state of the computer system and the data that it has collected about the walking steadiness of the user, thereby providing improved visual feedback. In some embodiments, the baseline condition is met when the computer system determines that the user's walking steadiness state has changed. For example, the computer system determines that the user's walking steadiness state is low and remains low, subsequently when the user's walking steadiness state changes from low to very low, the baseline condition is met. In some embodiments, as a result, the computer system does not repeatedly provide a notification for a low or very low steadiness state when the user's steadiness state is constant (e.g., does not get reduced).

In some embodiments, the duration over which the baseline walking steadiness state is determined over is a duration that transpired prior to the computer system (e.g., 600 and/or 651) being enabled (e.g., via input 610 and/or input 634) to provide notifications of walking steadiness state (e.g., prior to the feature being configured and/or enabled by the user on the computer system). Using information collected over a duration prior to the computer system being enabled to provide notifications of the walking steadiness state provides the user with feedback about the state of the computer system and the data that it has collected about the walking steadiness of the user, thereby providing improved visual feedback. In some embodiments, the baseline walking steadiness state and/or baseline walking steadiness metrics are based on movement data collected prior to the computer system being enabled to provide the user with feedback about the state of walking steadiness.

In some embodiments, the notification (e.g., 664, 668, 690, 692 and/or 696) associated with the walking steadiness state includes an indication of the walking steadiness state (e.g., that it is the first state). Providing an indication of the walking steadiness state provides the user with feedback about the state of the computer system and the data that it has collected about the walking steadiness of the user, thereby providing improved visual feedback. In some embodiments, the notification specifies that the walking steadiness state is low without specifying (without displaying) the walking steadiness score.

In some embodiments, the notification (e.g., 692 and/or 696) associated with the walking steadiness state includes an indication of a change in the walking steadiness state (e.g., indicates that the walking steadiness has improved, indicates that the walking steadiness has degraded). Providing an indication of a change in the walking steadiness state provides the user with feedback about the state of the computer system and the data that it has collected about the walking steadiness of the user, thereby providing improved visual feedback.

In some embodiments, movement data associated with movement (e.g., walking movement) of a user of the computer system includes data selected from the group consisting of walking symmetry (e.g., as illustrated at 682-4), length of stride (e.g., how far the user travels with each stride), speed of movement (e.g., as illustrated at 682-3) (e.g., speed of walking), step length (e.g., as illustrated at 682-2) (e.g., how far the user travels with one (or each) step, the difference between step lengths of different legs), double support time (e.g., as illustrated at 682-1) (e.g., based on duration of double support in the gait cycle, based on the time/duration when both feet are in contact with the ground simultaneously, and/or the time elapsed during two periods of double support in the gait cycle), or any combination thereof. In some embodiments, the computer system performs gait analysis on the movement data to determine the walking steadiness state of the user.

In some embodiments, the walking steadiness state is determined based on (e.g., based at least on) biometric data (e.g., as illustrated at 620) (e.g., height, weight, gender, and/or age) of the user of the computer system. In some embodiments, the computer system receives the biometric data via user input (e.g., on a touch-sensitive surface).

In some embodiments, the computer system (e.g., 600 and/or 650) receives one or more user inputs (e.g., 642, 666, 670, 694) (e.g., on a touch-sensitive surface of the computer system). In response to receiving the one or more user inputs, the computer system displays, via a display generation component (e.g., 601 and/or 651), a walking steadiness user interface (e.g., 604, 644) that includes an indication (e.g., 646, 648, 658, and/or 696) of the (e.g., current and/or most recent) walking steadiness state (e.g., first state). Displaying a walking steadiness user interface that includes an indication of the walking steadiness state provides the user with feedback about the state of the computer system and the data that it has collected about the walking steadiness of the user, thereby providing improved visual feedback.

In some embodiments, the one or more user inputs are received while the first set of conditions (e.g., and not the second set of conditions) is met. In some embodiments, the one or more user inputs includes activation of (e.g., tap input on and/or swipe input on) the notification associated with the walking steadiness state. In some embodiments, the one or more user inputs includes selection of an option (while the first set of conditions is met) to view a walking steadiness state (e.g., as part of a health application user interface).

In some embodiments, the one or more user inputs are received while the second set of conditions (e.g., and not the first set of conditions) is met. In some embodiments, the one or more user inputs includes selection of an option (while the second set of conditions is met) to view a walking steadiness state (e.g., as part of a health application user interface).

In some embodiments, the walking steadiness user interface includes a visual representation of a baseline walking steadiness state (e.g., data 641-1 in FIG. 6G; chart 646). Displaying a walking steadiness user interface that includes a visual representation of a baseline walking steadiness state provides the user with feedback about the state of the computer system and the data that it has collected about the walking steadiness of the user, thereby providing improved visual feedback.

In some embodiments, the walking steadiness user interface includes a history of (e.g., previous periods of time of) walking steadiness states (e.g., 646-1, 646) (e.g., as part of a chart or graphical representation and/or concurrently with the current or most recent walking steadiness state). Displaying a walking steadiness user interface that includes a history of walking steadiness state provides the user with feedback about the state of the computer system and the data that it has collected about the walking steadiness of the user, thereby providing improved visual feedback. In some embodiments, the computer system detects selection of (e.g., tap on) the history of walking steadiness states (e.g., the chart) and, in response, displays steadiness state for specific time (e.g., 7 day measurement period).

In some embodiments, the indication of the walking steadiness state (e.g., 662, 658, 696-1, 969-2) is concurrently displayed (as part of the walking steadiness user interface) with a representation (e.g., 660-1) of a first state of walking steadiness (e.g., low walking steadiness state and/or very low walking steadiness state, the user is not steady when walking) and a representation (e.g., 660-2) of a second state of walking steadiness (e.g., high walking steadiness state, the user is steady when walking) (and, optionally, a representation (e.g., 660-3) of a third state (e.g., very low steadiness)). Displaying multiple representations of different walking steadiness states provides the user with feedback about the different potential steadiness states that can be achieved, thereby providing improved visual feedback. In some embodiments, each representation of a walking steadiness state is displayed as a horizontal bar (e.g., graphical bars corresponding to each of okay, low, very low steadiness states). In some embodiments, the indication of the walking steadiness state is displayed overlaid on the representation of the respective state and is not overlaid on the on the other representations of walking steadiness states.

In some embodiments, a displayed position of the indication (e.g., 662) (e.g., a graphical element, such as a dot) of the walking steadiness state with respect to a respective representation of a steadiness state (e.g., 660-1, 660-2, 660-3) (e.g., the representation of the first state of walking steadiness or the second state of walking steadiness) indicates a walking steadiness score of the user with respect to a range of walking steadiness scores for the respective walking steadiness state. Displaying the indication with respect to the representations walking steadiness states provides the user with feedback about the state of the computer system and the data that it has collected about the walking steadiness of the user, thereby providing improved visual feedback. In some embodiments, the user's walking steadiness state is low and, as a result, the indication (e.g., a dot) is displayed overlaid on the representation of the first walking steadiness state. The position at which the indication is overlaid on (e.g., top of, middle of, or bottom of) the representation of the first walking steadiness state is based on where the user's walking steadiness is in the range of the first walking steadiness state.

In some embodiments, the walking steadiness user interface includes an indication (e.g., 655) (e.g., a textual and/or graphical) of a risk (e.g., over an (optionally) specified amount of time, such as the next 6 months or the next 12 months) of the user falling (e.g., a higher-than-average risk, a high risk). Displaying an indication of a risk of the user falling provides the user with feedback about the state of the computer system and the data that it has collected about the walking steadiness of the user, thereby providing improved visual feedback. In some embodiments, the risk of the user falling is determined based on one or more of: the current walking steadiness state, movement data associated with movement (e.g., walking movement) of a user of the computer system, and/or biometric data (e.g., height, weight, gender, and/or age) of the user of the computer system.

In some embodiments, the walking steadiness user interface includes (e.g., in a scrollable list) information (e.g., 674, 684, 686, 688) (e.g., videos, PDFs, health articles, links to applications) for improving walking steadiness. Displaying information for improving walking steadiness provides the user with feedback about what actions to take (e.g., exercises to perform) to change the user's walking steadiness, thereby providing improved visual feedback. In some embodiments, walking steadiness user interface includes an option to enable/disable notifications indicating state of walking steadiness. In some embodiments, walking steadiness user interface includes an indication that walking steadiness has improved (or degraded).

In some embodiments, the information for improving walking steadiness includes a video (e.g., 686, 688) (e.g., workout video) that incorporates (or multiple videos that each incorporate) one or more exercises directed to improving walking steadiness and one or more exercises not directed to improving walking steadiness. Displaying exercises for improving walking steadiness provides the user with feedback about what actions to take to improve the user's walking steadiness, thereby providing improved visual feedback. In some embodiments, the walking steadiness user interface includes videos (e.g., in-line) and/or links to videos that include walking steadiness exercises (e.g., one or more of: strength, balance, agility, reaction time, and/or turn exercises) within a set of exercises (e.g., traditional strength workout) to incentivize the user to perform the walking steadiness exercises.

In some embodiments, the computer system (e.g., 600 and/or 650) correlates changes in the walking steadiness state of the user with the information displayed for improving walking steadiness (e.g., workout videos). Correlating changes in the walking steadiness of the user with the displayed information enables the computer system to refine the information displayed for improving walking steadiness for the user, thereby providing improved visual feedback. In some embodiments, the computer system collects information to correlate improvements in a user's walking steadiness with the workouts performed by the user (and/or recommended to the user). This information may optionally be used to provide the user (or other users) with exercise recommendations that have more significant positive results. In some embodiments, the computer system dynamically adjusts the underlying algorithm (e.g., for determining the walking steadiness state and/or for recommending exercises, videos, and/or literature) based on the user's walking steadiness. As the user's walking steadiness changes, different exercises, videos, and/or literature may be recommended. In one implementation, the computer system can recommend different exercises and videos to different users based on each user's individual walking steadiness. Further, in some implementations, the computer system can correlate exercises, walking steadiness, and improvement, and provide recommendations as the user progresses in walking steadiness.

In some embodiments, the computer system (e.g., 600 and/or 650) determines whether the user has (e.g., within a predefined amount of time, such as 2 weeks or 3 weeks) performed (e.g., completed) (e.g., based on a determination of articles displayed and/or duration/portion of videos displayed) recommended (or a specific portion of recommended) exercises (e.g., as recommended in the information for improving walking steadiness). In accordance with a determination that the user has not (e.g., within a predefined amount of time, such as 2 weeks or 3 weeks) performed (e.g., has not completed) the recommended (or a specific portion of recommended) exercises, the computer system provides a notification (e.g., displaying an alert on a display) reminding the user to perform the exercises (in some embodiments, prompting the user to perform the exercises and/or notifying the user of incomplete exercises). In accordance with a determination that the user has performed (e.g., has completed) the recommended (or a specific portion of recommended) exercises, the computer system forgoes providing the notification reminding the user that the user has not performed the exercises. Providing a notification to the user when the user has not performed the recommended exercises provides the user with feedback about what actions to take to improve the user's walking steadiness and the state of the computer system (e.g., that it has not detected the user performing the exercises), thereby providing improved visual feedback. In some embodiments, the computer system analyses the exercises that the user has performed (e.g., based on a determination of articles displayed and/or duration/portion of videos displayed) to determine whether (and which) additional exercises would benefit the user. In some embodiments, the additional exercises are presented to the user (e.g., as part of articles and/or videos in the walking steadiness user interface). In some embodiments, the computer system provides the user with a notification (e.g., by displaying, via a display generation component) that one or more recommended (or accessed) exercises have not been completed (e.g., in accordance with a determination that the user's walking steadiness state has not improved).

In some embodiments, the computer system enables the user to (e.g., by providing a user interface for) design workouts by displaying (e.g., as part of a recommendation) segments (such as from a categories of segments—strength, balance, agility, reaction times, turns) that the user can perform to help improve walking steadiness. The computer system enables the user to user choose the segments and sequence to create a workout tailored to the user. In one implementation, the computer system surfaces recommended segments based on the individual user's walking steadiness and may surface different recommended segments for different users.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve the delivery to users of walking steadiness content or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content that is of greater interest to the user. Accordingly, use of such personal information data enables users to have calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining walking steadiness, the present technology can be configured to allow users to select to “opt in” or “opt out” of participation in the collection of personal information data during registration for services or anytime thereafter. In another example, users can select not to provide some movement data for determining walking steadiness. In yet another example, users can select to limit the length of time walking data is maintained or entirely prohibit the development of a walking steadiness baseline. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, walking steadiness data or a walking steadiness baseline can be determined by inferring measurements based on non-personal information data or a bare minimum amount of personal information, such as the content being requested by the device associated with a user, other non-personal information available to the device, or publicly available information.

What is claimed is:

1. A computer system configured to communicate with one or more output generation components and one or more input devices, comprising:
one or more processors; and
memory storing one or more programs configured to be executed by the one or more processors, the one or more programs including instructions for:
detecting, via the one or more input devices, movement data associated with movement of a user of the computer system;
determining a walking steadiness state based on the detected movement data; and
in response to determining the walking steadiness state:
in accordance with a determination that a first set of conditions is met, outputting, via the one or more output generation components, a notification associated with the walking steadiness state; and
in accordance with a determination that a second set of conditions, different from the first set of conditions, is met, forgoing outputting a notification associated with the walking steadiness state.

2. The computer system of claim 1, wherein the first set of conditions includes a state condition that is met when the walking steadiness state is a first state of walking steadiness that corresponds to a first range of walking steadiness scores and the second set of conditions includes a state condition that is met when the walking steadiness state is a second state of walking steadiness that corresponds to a second range of walking steadiness scores that is different from the first range of walking steadiness scores.

3. The computer system of claim 1, wherein the first set of conditions includes a duration condition that is met when the walking steadiness state is maintained in a first state of walking steadiness for a first duration.

4. The computer system of claim 1, wherein the second set of conditions includes a duration condition that is met when the walking steadiness state has been maintained in a second state of walking steadiness for a second duration.

5. The computer system of claim 1, the one or more programs further including instructions for:
subsequent to outputting, via the one or more output generation components, the notification associated with the walking steadiness state and in accordance with a determination that the walking steadiness state has remained in a first state of walking steadiness for a third duration, providing a second notification that indicates that the walking steadiness state is the first state.

6. The computer system of claim 1, wherein the first set of conditions includes a baseline condition that is met when the walking steadiness state is different from a baseline walking steadiness state, wherein the baseline walking steadiness state is a walking steadiness state of the user determined over a duration.

7. The computer system of claim 6, wherein the duration over which the baseline walking steadiness state is determined over is a duration that transpired prior to the computer system being enabled to provide notifications of walking steadiness state.

8. The computer system of claim 1, wherein the notification associated with the walking steadiness state includes an indication of the walking steadiness state.

9. The computer system of claim 1, wherein the notification associated with the walking steadiness state includes an indication of a change in the walking steadiness state.

10. The computer system of claim 1, wherein movement data associated with movement of a user of the computer system includes data selected from the group consisting of walking symmetry, length of stride, speed of movement, step length, double support time, or any combination thereof.

11. The computer system of claim 1, wherein the walking steadiness state is determined based on biometric data of the user of the computer system.

12. The computer system of claim 1, the one or more programs further including instructions for:
receiving one or more user inputs; and
in response to receiving the one or more user inputs, displaying, via a display generation component, a walking steadiness user interface that includes an indication of the walking steadiness state.

13. The computer system of claim 12, wherein the one or more user inputs are received while the first set of conditions is met.

14. The computer system of claim 12, wherein the one or more user inputs are received while the second set of conditions is met.

15. The computer system of claim 12, wherein the walking steadiness user interface includes a visual representation of a baseline walking steadiness state.

16. The computer system of claim 12, wherein the walking steadiness user interface includes a history of walking steadiness states.

17. The computer system of claim 12, wherein the indication of the walking steadiness state is concurrently displayed with a representation of a first state of walking steadiness and a representation of a second state of walking steadiness.

18. The computer system of claim 17, wherein a displayed position of the indication of the walking steadiness state with respect to a respective representation of a steadiness state indicates a walking steadiness score of the user with respect to a range of walking steadiness scores for the respective walking steadiness state.

19. The computer system of claim 12, wherein the walking steadiness user interface includes an indication of a risk of the user falling.

20. The computer system of claim 12, wherein the walking steadiness user interface includes information for improving walking steadiness.

21. The computer system of claim 20, wherein the information for improving walking steadiness includes a video that incorporates one or more exercises directed to improving walking steadiness and one or more exercises not directed to improving walking steadiness.

22. The computer system of claim 20, the one or more programs further including instructions for:
correlating changes in the walking steadiness state of the user with the information displayed for improving walking steadiness.

23. The computer system of claim 20, the one or more programs further including instructions for:
determining whether the user has performed recommended exercises;
in accordance with a determination that the user has not performed the recommended exercises, providing a notification reminding the user to perform the exercises; and
in accordance with a determination that the user has performed the recommended exercises, forgoing providing the notification reminding the user that the user has not performed the exercises.

24. A non-transitory computer-readable storage medium storing one or more programs configured to be executed by one or more processors of a computer system that is in communication with one or more output generation components and one or more input devices, the one or more programs including instructions for:
detecting, via the one or more input devices, movement data associated with movement of a user of the computer system;
determining a walking steadiness state based on the detected movement data; and
in response to determining the walking steadiness state:
in accordance with a determination that a first set of conditions is met, outputting, via the one or more output generation components, a notification associated with the walking steadiness state; and
in accordance with a determination that a second set of conditions, different from the first set of conditions, is met, forgoing outputting a notification associated with the walking steadiness state.

25. A method, comprising:
at a computer system that is in communication with one or more output generation components and one or more input devices:
detecting, via the one or more input devices, movement data associated with movement of a user of the computer system;
determining a walking steadiness state based on the detected movement data; and
in response to determining the walking steadiness state:
in accordance with a determination that a first set of conditions is met, outputting, via the one or more output generation components, a notification associated with the walking steadiness state; and
in accordance with a determination that a second set of conditions, different from the first set of conditions, is met, forgoing outputting a notification associated with the walking steadiness state.
